(19) United States
(12) Patent Application Publication
(10) Pub. No.: US 2010/0129445 A1
(43) Pub. Date: May 27, 2010

(54) GASTRORETENTIVE SYSTEM COMPRISING AN ALGINATE BODY

(75) Inventors: Bodo Asmussen, Bendorf (DE); Christiane Schiller, Stralsund (DE); Werner Weitschies, Neuenkirchen (DE)

Correspondence Address: D. PETER HOCHBERG CO. L.P.A., 1940 EAST 6TH STREET, CLEVELAND, OH, 44114, US

(73) Assignee: LTS LOHMANN THERAPIE-SYSTEME AG, ANDERNACH (DE)

(21) Appl. No.: 12/451,926

(22) PCT Filed: May 27, 2008
(86) PCT No.: PCT/EP2008/004188

§ 371 Date: Dec. 4, 2009

(30) Foreign Application Priority Data
Jun. 4, 2007 (DE) 10-2007-026-037.9

(51) Int. Cl. A61K 9/22; A61K 33/00; A61K 33/30; A61K 33/06

(52) U.S. Cl. 424/468; 424/722; 424/641; 424/682

(57) ABSTRACT

Gastroretentive systems which have at least one release device for at least one active pharmaceutical ingredient and at least one swelling body that is connected to the release device.

### CROSS-REFERENCE TO RELATED APPLICATIONS

This application is a National Stage application of International Application No. PCT/EP2008/004188, filed on May 27, 2008, which claims priority of German application number 10 2007 026 037.9, filed on Jun. 4, 2007, both of which are incorporated herein by reference in their entireties.

### BACKGROUND OF THE INVENTION

1. Field of the Invention

The invention relates to an orally applicable gastroretentive system for the controlled, continuous release of at least one active pharmaceutical substance (=active pharmaceutical ingredient) in the stomach, which comprises at least one release device and at least one swelling body that is firmly connected to the release device, wherein said release device(s) and said swelling body/bodies are able to function independently from each other.

2. Description of the Prior Art

One aim of developing medications is to provide forms of medication by means of which it is possible to maintain an active ingredient level in the patient's body that will remain constant for several hours. With rapidly disintegrating tablets, however, this cannot be achieved since these tablets release the active ingredient contained therein all at once. For this reason, tablet-shaped medications have been developed which are capable of continuously releasing the active pharmaceutical ingredient contained therein in a controlled manner and over a prolonged period of time.

Thus, U.S. Pat. No. 5,296,233 describes a capsule-like medicament comprising a dual subcoating, said medicament comprising a solid, active ingredient-containing capsule core and a dual subcoating composition. The dual subcoating composition comprises an initial subcoating, which comprises a water-soluble, film-forming polymer, e.g. povidone, and is applied to the capsule core, and a second subcoating which comprises a mixture of at least one water-soluble, film-forming polymer and a hydrophobic plasticizer, e.g. castor oil. Furthermore, this medicament has a smooth, uniform and substantially bubble free outer coating to enable the medicament to be easily swallowed despite its comparatively large volume.

U.S. Pat. No. 4,983,401 describes a sustained-release pharmaceutical preparation utilizing a pH-controlled diffusion membrane composed of a pH-sensitive film-forming polymer. The film-forming polymer may contain phthalic acid groups which are attached, with one of their carboxyl groups, to the starting polymer via an ester bond, whereas the second carboxyl group remains free as a free acid so that the modified film-forming polymer is hydrophobic at low pH and hydrophilic at higher pH.

EP 0 259 219 A2 describes a press-coated tablet having a central opening through which the active ingredient is released from an erodible tablet core to the outside. The thickness of the tablet core increases from the central opening towards the periphery, which leads to the increase in the distance between the erosion front and the opening, which occurs as the active substance is being released, being compensated by an increase in surface area.

From EP 0 542 364 A1, there is known a device for the controlled release of at least one active ingredient into a fluid medium, said device being present in the form of a tablet. The device comprises a covering which is impermeable to both the active substance and the medium. Said covering has at least one opening therein and defines a cavity, said cavity being filled by a core comprising the active substance, with said core extending as far as said opening. The geometric shape and the formulation of the tablet are such that the active substance can be released at a constant rate over a significant period of time.

EP 0 779 807 A1 discloses a press-coated tablet for the controlled release of active substances. Said tablet has a core which can be eroded and contains at least one active substance, and has a largely erosion-resistant coating layer that forms a covering and has at least one opening. Said core is formed so as to have a tapered end region that is pointed or narrow, and is placed in the tablet such that its pointed or narrow end region extends as far as the outer edge of the press-coated tablet and at that place interrupts the mass of the coating layer surrounding the core so that the opening at the tablet's outer edge is formed.

EP 0 797 429 A1 describes an osmotic device for the continuous release of active substances in the fluids of the gastro-intestinal tract, wherein the active substance is released through an outlet opening in the outer membrane of the device. The outlet opening is situated at a location of the outer membrane which is recessed relative thereto so that it cannot come into contact with the surface of the mucous membrane.

With these orally applied dosage forms, too, it is disadvantageous that the plasma level of the active pharmaceutical ingredient can be different from one patient to another and is difficult to predict. The reason for this may be that the absorbability of the active pharmaceutical ingredient varies strongly from one region of the gastrointestinal tract to another and that the administered dosage form is transported through the gastrointestinal tract at a different rate in different patients. A term commonly used in connection with the changing absorbability along the gastrointestinal tract is “absorption windows.” For example, some active pharmaceutical ingredients are preferably absorbed in sections of the intestine that are in the vicinity of the pylorus. Immediately adjacent to the pylorus there is the approximately 30-cm-long duodenum, which for a large number of active pharmaceutical ingredients provides the highest absorption capacity. In the lower sections of the intestine, which are at a greater distance to the pylorus, these active pharmaceutical ingredients are as a rule absorbed only in very low amounts. Since it is at present not possible to make reliable statements on the transit rate of an active pharmaceutical ingredient in patients, it cannot be predicted when an active pharmaceutical ingredient after oral administration thereof will be in a region of the gastrointestinal tract that is favourable for the absorption of the active pharmaceutical ingredient contained therein.

To enable a long-lasting, constant plasma level of an active pharmaceutical ingredient that is absorbed in the gastrointestinal tract and preferably has its absorption window in the duodenum or upper portion of the small intestine, solid active pharmaceutical ingredients having a prolonged retention time in the stomach represent a promising approach to finding a solution to the above-described problems. Such systems are also referred to as “gastroretentive systems.”

Gastroretentive systems, i.e. forms of medication having a retention time in the stomach which is longer than that of capsules or tablets, are known as such. Gastroretentive systems not containing an active pharmaceutical ingredient serve to at least partially fill the stomach to produce a feeling of satiety and thereby achieve a reduction in weight. Active ingredient-containing gastroretentive systems enable a retarded release of active pharmaceutical ingredients in the stomach.

Gastroretentive systems should largely correspond to conventional solid oral forms of medication as regards the directions for patients on how to take the medication so that the known habits of taking medications can be maintained. They should have an acceptable size and should be easy to swallow. In addition, gastroretentive systems should remain in the stomach for sufficiently long and there release the active pharmaceutical ingredient contained in said systems in a controlled manner. Having fulfilled its task, the gastroretentive system should either be decomposed in the gastrointestinal tract or leave it safely via a natural route.

To this end, gastroretentive devices have been developed which have a lower density than the contents of the stomach. These devices are to float on the gastric juice or the gastric contents due to their buoyancy. WO 02/85332 A1, for example, describes devices that have a high proportion of lipophile substances having a low density.

EP 0 326 816 A discloses a floatable active ingredient dosage form which ensures a long retention time in the gastrointestinal tract and wherein cavities are enclosed by at least one structural element. More particularly, the structural element may be a foamed or microporous polymer matrix, for instance of polyolefin, polyamide, polyester, polystyrene, polyacrylate, polytetrafluoroethylene (PTFE), polyvinylchloride (PVC), polyvinylidene chloride or polysiloxane, optionally in the form of a foldable or rollable film, a tablet core, or in layered form. Alternatively, the structural elements can be hollow particles of, for example, glass or ceramics that are embedded in a matrix composition containing active ingredient, particularly for use in capsules. The structures may be provided with a membrane controlling the release of active substance.

Another approach to achieving a longer retention time in the stomach was to develop gastroretentive systems which because of their size or shape are not able to pass through the pylorus and can therefore not leave the stomach. These devices are generally present in a compressed form and develop their intended size only upon contact with gastric juice.

WO 02/00213 A1, for example, discloses a gastroretentive form of medication with a rapidly expanding preparation that consists of a very rapidly disintegrating substance, tannic acid, and at least one hydrogel.

WO 2005/079384 A2 also discloses an expansible gastroretentive device, said device comprising a dried polysaccharide gel which may contain an active pharmaceutical ingredient.

WO 01/97783 A1 addresses the influence of the size and swelling properties of a gastroretentive system on the retention time thereof in the stomach.

In addition, gastroretentive systems are known that are retained in the stomach for a prolonged period due to an increase in volume caused by generation of gas, and likewise float on the gastric contents.

An example of this is a dosage form according to U.S. Pat. No. 4,996,058, wherein the active ingredient is contained in a closed bag made of a hydrophilic membrane, along with a compound that generates carbon dioxide or nitrogen on coming into contact with gastric juices. In this gastroretentive system, the active ingredient is dissolved by the gastric juice entering the bag and is released via the membrane of the bag, which controls the release of the active ingredient.

Systems of this type are described, for example, in U.S. Pat. No. 4,207,890 and DE 44 19 818 A1. Their mode of action is based essentially on filling a bag with active ingredient, which bag dissolves within a defined period of time and is released to the environment in a controlled manner. The volume increase is achieved in that there also are suitable gas-generating substances (such as sodium hydrogen carbonate) or gas-generating substance mixtures in the bag which by a chemical reaction release a gas, e.g. CO₂, when the hydrochloric acid-containing gastric juice enters the bag. In the process, the bag, which consists of a membrane, is blown up, as it were, and reaches a size that prevents the system's passage to the duodenum.

When the gastric juice enters the bag, active ingredient is released simultaneously. The release characteristics of the active ingredient can be controlled by its shape, e.g. an active ingredient being present as a microencapsulated particle, or by the properties of the membrane. Once the active ingredient diffuses through the membrane and has thereby reached the gastric contents, it can be absorbed via the gastric mucous membrane or the intestinal wall. Since the system remains in the stomach for a prolonged period of time, the controlled active ingredient release in the stomach will take place for this prolonged period, preferably up to 24 hours.

Gastroretentive devices containing carbon dioxide-generating components are also described in WO 03/011255 A1 and US 2006/003003 A1.

Functioning of the floating system, however, depends on the amount of liquid present in the stomach. To enable such forms of medication to float in the first place, there must be a minimum amount of gastric contents or gastric juice in the stomach. However, in fasting probands there was only an average of 20 to 50 ml of liquid in the stomach. Hence, in fasting patients it will hardly be possible to realise stable floating and hence hardly be possible to avoid, by means of buoyancy, that the system leaves the stomach. This is also likely to be a reason for the unreliability of systems of that type.

Another approach was adopted by separating the task of releasing the active pharmaceutical ingredient from the task of ensuring the residence of the system in the stomach. Such dual systems, consisting of an active ingredient-containing matrix and a swelling layer, are described in US 2005/0019409 A1 and US 2006/0013876 A1, for example. A disadvantage of such systems, however, is that the release of the active ingredient from the matrix is diffusion-controlled and may, as the case may be, depend on the environmental conditions such as gastric contents, pH value, ionic strength and pressure.

### SUMMARY OF THE PRESENT INVENTION

The invention is based on the object of developing a gastroretentive system for the controlled release of active pharmaceutical ingredient over a prolonged period of time which is free of the disadvantages of the gastroretentive systems known from the state of the art.

The object is achieved by a gastroretentive system which comprises two elements that function independently from each other, but which are firmly connected to one another. The first element (element A) is at least one swelling body which prolongs the retention time of the system in the stomach and which is preferably based on a sodium alginate. The second element (element B) is at least one release device for the active pharmaceutical ingredient which enables a controlled release of said active pharmaceutical ingredient; for example, an osmotically controlled or an erosion-controlled release.

Hence, the subject-matter of the invention is a gastroretentive system which comprises at least one swelling body and at least one device for the release of at least one active pharmaceutical ingredient, said swelling body/bodies and said release device(s) being firmly connected to one another but being able to function independently from one another.

Hence, the gastroretentive system according to the invention may comprise embodiments having only one swelling body, but also embodiments having several swelling bodies. Likewise, it may comprise embodiments having only one release device or embodiments having several release devices by means of which one and the same active pharmaceutical ingredient or different active pharmaceutical ingredients can be administered. As a matter of course, the statements made in the following, relating to swelling bodies and release device apply to all embodiments of the gastroretentive system according to the present invention, even where expressions in the singular form are used in the following.

### BRIEF DESCRIPTION OF THE DRAWINGS

FIG. 1 is a diagram illustrating the relative change of mass of sodium alginate-based swelling bodies in an aqueous medium having a pH of 3.

FIG. 2 is a diagram illustrating the relative change of mass of sodium alginate-based swelling bodies in an aqueous medium having a pH of 4.5.

FIG. 3 is a diagram illustrating the relative change of mass of a sodium alginate-based swelling bodies in an aqueous medium whose pH is increased stepwise.

### DETAILED DESCRIPTION OF THE PRESENT INVENTION

The swelling body ensures a longer retention time of the system in the stomach by swelling in the stomach after its administration. Preferably, the swelling body is based on sodium alginate, which is characterised by its good swelling properties. After its introduction in the stomach, the swelling body can develop its full size. In the environment present in the human intestine, the swelling body quickly dissolves so that after it has been emptied from the stomach, its accumulation in the intestine and thus a potentially threatening intestinal obstruction can be avoided.

In a preferred embodiment, a pharmaceutically acceptable calcium salt is added to the sodium alginate, or a mixture of sodium alginate and calcium alginate is used.

Sodium alginate exhibits sufficient stability only at pH values from 1 to 2, but in the environment of the human intestine it dissolves relatively quickly. Through the addition of a calcium salt, the swelling properties of the swelling body or sodium alginate body are stabilised at higher ph values than those aforementioned. Surprisingly, the disintegration properties of the swelling body required for reasons of safety are maintained in the “neutral” environment of the intestine even if calcium ions or calcium alginate have/has been added thereto.

The addition of calcium ions may in principal take place by addition of any pharmaceutically acceptable calcium salt or any mixture of two or more of such calcium salts. Pharmaceutically acceptable calcium salts are, for example, calcium acetate, calcium aspartate, calcium carbonate, calcium chloride, calcium citrate, calcium cyclamate, calcium folinate, calcium gluconate, calcium glutamate, calcium lactate, calcium lactate gluconate, calcium phosphate and calcium sulfate.

The proportion of calcium ions contained in the swelling body may be between 0.1 and 10%-wt., relative to the mass of the swelling body. It is preferably between 0.3 and 8%-wt., more preferably between 0.5 and 5%-wt. Most preferably, the swelling body has a proportion of calcium ions of 0.6 to 2%-wt.

As an alternative to or in addition to adding calcium ions, it is also possible to use zinc ions and/or aluminium ions in the form of pharmaceutically acceptable salts.

Pharmaceutically acceptable zinc salts are, for example, zinc acetate, zinc aspartate, zinc bishydrogenaspartate, zinc chloride and zinc gluconate. Aluminium salts that can be used from a pharmaceutical point of view are, for example, aluminium hydroxide, algedrate (47-60% aluminium oxide (Al₂O₃)) and aluminium phosphate.

The proportion of zinc salt(s) or aluminium salt(s) contained in the swelling body is preferably between 0.1 and 30%-wt., more preferably between 1 and 25%-wt., and most preferably between 5 and 15%-wt., relative to the mass of the swelling body.

It is also possible to use mixtures of sodium alginate with further polymers also having swelling properties, provided that the pH-dependent disintegration properties of the swelling body are maintained. For example, mixtures of sodium alginate with croscarmellose sodium, polycarbophil (polyacrylate crosslinked with divinyl glycol), polyethylene oxide and/or cellulose derivatives can be mentioned, addition of croscarmellose sodium being particularly preferred. Among the cellulose derivatives, non-water-soluble cellulose derivatives are preferred, especially ethyl cellulose and hydroxypropyl methyl cellulose.

The proportion of other polymers is preferably 1 to 30%-wt., more preferably 3 to 20%-wt., and most preferably 5 to 15%-wt., relative to the mass of the swelling body.

To produce the swelling body, further suitable excipients can be employed additionally, such as, for example, flow regulators, lubricants or glidants, fillers, binders and/or antiadherents. As fillers sugar derivatives, sugars such as sucrose or glucose, sugar substitutes such as xylitol or sorbitol can be used. Lactose or microcrystalline cellulose are used with particular preference. As binders polyvinyl pyrrolidone, gelatine, methyl cellulose, ethyl cellulose, gum arabic, tragacanth, polyethylene glycol and starch derivatives may be used. Glidants suitable for use are magnesium stearate, calcium stearate, calcium behenate, glycerol monostearate, stearic acid and its salts, waxes, highly dispersed silicon dioxide and hydrogenated vegetable fats.

Furthermore, substances may be used that are able to influence the pH locally in the dosage form, e.g. citric acid, polycarbophil or algedrate.

The release device of the gastroretentive system according to the invention enables a release of the active pharmaceutical ingredient contained therein at a constant rate over a prolonged period of time, preferably during the entire period of application. The release of the active pharmaceutical ingredient takes place independently from environmental conditions which would interfere with a diffusion-controlled active pharmaceutical ingredient release; for example osmotically controlled or erosion-controlled.

The swelling body and the release device are firmly connected to each other. To achieve this, the swelling body and the release device may be compressed with each other or glued to each other, so that the gastroretentive system may be provided in the form of a bilayer or multilayer tablet or in the form of a press-coated tablet, for example.

For the adhesive, which serves to connect the swelling body and the release device firmly to each other, physiologically tolerated, pharmaceutically acceptable adhesives are used. Polymers having adhesive properties may be used as adhesives. These include, for example, acrylates, methyl methacrylate polymers, dextrin-based adhesives, acrylate-vinyl acetate-based adhesives, carboxyvinyl polymers, cellulose acetates and ethyl celluloses.

However, there is also a possibility of arranging the swelling body and the release device within a common covering which is permeable to the gastric juice as well as to the active pharmaceutical ingredient, for example within a net.

In one embodiment of the gastroretentive system according to the invention, the release device is an osmotic system that may be present in the form of a single-chamber system or as a multi-chamber system. It may also be present in the form of an embodiment wherein spatially separated regions for the active pharmaceutical ingredient and for the osmotically active substance are not separated from each other by a membrane.

If the release device is designed as a single-chamber system, an active pharmaceutical ingredient-containing core is surrounded by a semipermeable polymer membrane which has a small opening. The active pharmaceutical ingredient-containing core of this release device is generally pressed from a mixture of active pharmaceutical ingredient and excipients. The polymer membrane may be applied to the core by a spraying process with which the polymer is applied to the core with further excipients as a solution or dispersion and then dried. The outlet opening in the polymer membrane may be produced by a laser beam.

After the peroral administration of a release device of this type as a component of the gastroretentive system according to the invention, the water contained in the gastric juice diffuses from the polymer membrane into the core containing the active pharmaceutical ingredient. The active pharmaceutical ingredient and/or an osmotically active excipient intended for this purpose begin to dissolve in the fluid that has entered the core. By this means, an increased osmotic pressure compared with the external medium results in the interior of the release device, which results in the active ingredient-containing solution being pushed outwards through the outlet opening. Because the rediffusion of further liquid through the polymer membrane into the core takes place continuously, pressure equalization due to escape of active pharmaceutical ingredient solution takes place with great uniformity, as long as active ingredient is present in the core. The rate of active ingredient release can be adjusted by means of the composition, condition and distension of the membrane and also by means of the solubility of the constituents of the core.

If an active pharmaceutical ingredient is to be released that has only limited solubility in the gastric fluid water penetrating the membrane and therefore has insufficient osmotic action, one or more osmotically active substances may be added to the active pharmaceutical ingredient. Preferably, the substances used as osmotically active additives are salts, such as sodium chloride, sodium carbonate, sodium sulfate, sodium sulfite, potassium sulfate, potassium chloride, acidic potassium phosphate (KH₂PO₄), calcium carbonate, calcium sulfate, calcium lactate, magnesium sulfate, magnesium chloride, lithium chloride, lithium sulfate, D-mannite, urea, inosite, tartaric acid, cane sugar, raffinose, glucose or α-D-lactose monohydrate. Provided the active pharmaceutical ingredient itself has sufficient osmotic activity, the addition of osmotically active substances can be dispensed with.

The polymer membrane of the release device for osmotically controlled active pharmaceutical ingredient release is semipermeable. This means that it is permeable to water but essentially impermeable to dissolved substances.

The materials which can be used for the production of semipermeable membranes for release devices by means of which the active pharmaceutical ingredient can be released in an osmotically controlled manner include, for instance, cellulose acetate, cellulose triacetate, agar acetate, amylose triacetate, β-glucan acetate, β-glucan triacetate, acetaldehyde dimethyl acetate, cellulose acetate methyl carbamate, cellulose acetate succinate, cellulose acetate dimethylaminoacetate, cellulose acetate ethyl carbonate, cellulose acetate chloroacetate, cellulose acetate ethyl oxalate, cellulose acetate methyl sulfonate, cellulose acetate butyl sulfonate, cellulose ether, cellulose acetate propionate, poly(vinyl methyl)ether copolymers, cellulose acetate diethylaminoacetate, cellulose acetoacetate, cellulose acetate laurate, methyl cellulose, cellulose acetate-p-toluene sulfonate, triacetate of gum arabic, cellulose acetate with acetylated hydroxy ethyl cellulose, hydroxylated ethylene vinyl acetate, polymeric epoxides, copolymers of an alkylene oxide and alkyl glycidyl ether.

Peroral osmotic therapy systems, however, can have the disadvantage of an injury potential for the gastric mucous membrane in addition to the advantage of control of the release rate. In this case, the injury potentials of the active compounds and excipients, which are often released in highly concentrated form, can be significantly increased as a result of the focusing through the small outlet opening, so that the intestinal wall is seriously injured at certain points.

Advantageously, in this case the release device for osmotically controlled active pharmaceutical ingredient release is configured such that the outlet opening compared with areas of the device designed to maintain a distance in relation to the mucous membrane surface, is arranged in a position of the external membrane at a distance from the surface of the mucous membrane which, relatively thereto, is further away.

In this connection, the release device can have hollows, concave curvatures, curved or angled axes, annular shaping or other design features in which direct or close contact of the outlet opening with the surface of the mucous membrane lining the stomach is not possible. The outlet opening for the active pharmaceutical ingredient solution is thereby compulsorily positioned at a distance from surface regions of the gastric wall because a distance is maintained between the outlet opening and the part of the adjacent gastric wall. This distance results in the active pharmaceutical ingredient solution not meeting a small area of the gastric mucous membrane corresponding to the outlet opening in the same high concentration in which it passes through the outlet opening, but meeting a larger area thereof in dilute form. After administration of a gastroretentive system according to the invention comprising a release device designed as above-described, active pharmaceutical ingredients or excipients having a potential to injure the mucous membrane reach the gastric mucous membrane only after prior dilution and can only damage the latter significantly less—if at all—than conventional osmotic therapy systems.

In the embodiments of the gastroretentive system according to the invention which have a release device comprising a multi-chamber system for osmotically controlled active pharmaceutical ingredient release, the active pharmaceutical ingredient preparation and the osmotically active substance are present in separate chambers within a common covering, with the chamber that contains the osmotically active substance not having an opening whereas the active pharmaceutical ingredient-containing chamber has an opening for exit of the active pharmaceutical ingredient, which opening leads outwards and optionally penetrates said common covering. The chamber containing the osmotically active substance is, at least in a region thereof, defined by a semipermeable membrane. The boundary of the active pharmaceutical ingredient-containing chamber, too, has to be flexible at least in a region thereof. After application of such a release device, the liquid entering the chamber with the osmotically active substance leads to a volume enlargement of that chamber. As a result of its expansion, the active pharmaceutical ingredient preparation is pressed out of its compartment through the opening and thereby enters the stomach.

In another embodiment, in which the active pharmaceutical ingredient release is erosion-controlled, the system according to the invention is structured like a press-coated tablet wherein the swelling body forms the coating layer which surrounds the tablet core. The tablet core consists of an erodible mass which contains the active pharmaceutical ingredient. The coating layer surrounding the tablet core has an opening through which active pharmaceutical ingredient is able to exit. The release rate at which the active pharmaceutical ingredient is released from the tablet core constituting the release device of this system can be adjusted by means of the shape and size of said tablet core, for example by forming an erosion front that becomes larger as the distance to the opening increases, as well as by means of its solubility/erosion rate (depending on the swelling behaviour/solubility of the polymers employed for the matrix).

The term “erosion” has become established in pharmaceutical technology to denote any processes in which solid matter masses are “carried away”. It is not critical in this connection whether the mass reduction of the solid body is brought about by a dissolution of solid components or by a chemical decomposition taking place initially in which, for example, long polymer chains are cleaved into more readily soluble oligomers, monomers or other degradation products.

The erodible mass consists of physiologically acceptable polymers or wax-like substances and, if required, further pharmaceutical excipients. Examples of such polymers are polysaccharides such as gums, starch derivatives or cellulose derivatives, polyacrylates and polymethacrylates, polylactides, polyglycolides, polyoxyethylenes and polyoxypropylenes, proteins, polyvinyl alcohol, polyvinyl acetate, polyvinyl chloride or polyvinyl pyrrolidone. Wax-like substances are, for example, hydrogenated castor oil or cetyl stearyl alcohol. Further pharmaceutical excipients may be selected from the groups of the stabilisers, solubilisers, tensides, fillers, plasticisers, hydrophilising agents, pigments or dyes, substances for adjusting the pH value, flow regulators, antiadherents, lubricants etc.

The proportion of the individual components must be adjusted according to the compatibility and the intended rate of erosion.

The invention also encompasses embodiments for erosion-controlled active pharmaceutical ingredient release wherein the release device can be present in the form of one or more layers.

The gastroretentive system according to the invention may be present as a single-layered or multilayered tablet, or as a press-coated tablet. In a preferred embodiment, it has a coating that disintegrates in the stomach, or it is present in the form of a capsule whose shell disintegrates in the stomach. The purpose of this coating or shell is to at least facilitate swallowing of the gastroretentive system.

The gastroretentive system according to the present invention specifically has the advantage that it is another structural element which ensures that the system is sorted back in the stomach than that effecting the continuous release of active ingredient. By this means it can be guaranteed that the backsorting mechanism of the stomach which is in the digestion phase will retain the gastroretentive system according to the invention as “a nourishment component not yet having been sufficiently reduced in size” and prevent it from being further transported into the small intestine before the end of the period intended for the release of the active pharmaceutical ingredient. The retention time of the gastroretentive system in the stomach can thus extend for the intended duration of the release of the active pharmaceutical ingredient, which should be at least 4 hours but not exceeding 24 hours, and should preferably be between 6 and 14 hours.

### EXAMPLES

### Example 1

Preparation of a swelling body

| Ingredient | Proportion |
|---|---|
| Sodium alginate | 59.1%-wt. |
| Microcrystalline cellulose | 39.4%-wt. |
| Magnesium stearate | 1.0%-wt. |
| Highly dispersed silicon dioxide | 0.5%-wt. |

Sodium alginate was granulated with the microcrystalline cellulose by means of the polyvinyl pyrrolidone solution (KOLLIDON®30 in ethanol) as binding agent. Subsequent to drying, the resultant granulate was classified (grain size 300 to 800 nm). This mixture, which is referred to as the inner phase, was equipped with the outer phase, consisting of magnesium stearate and highly dispersed silicon dioxide.

Subsequently the recipe was compressed by means of a tableting press into biplanar tablets having a diameter of 13 mm and a mass of 600 mg.

### Example 2

Preparation of a swelling body

| Ingredient | Proportion |
|---|---|
| Sodium alginate | 58.2%-wt. |
| Lactose | 38.8%-wt. |
| Calcium carbonate | 1.5%-wt. |
| Magnesium stearate | 1.0%-wt. |
| Highly dispersed silicon dioxide | 0.5%-wt. |

Sodium alginate was granulated with lactose and calcium carbonate by means of the polyvinyl pyrrolidone solution (KOLLIDON®30 in ethanol) as binding agent. Subsequent to drying, the resultant granulate was classified (grain size 300 to 800 nm). This mixture, which is referred to as the inner phase, was equipped with the outer phase which consisted of magnesium stearate and highly dispersed silicon dioxide.

Subsequently the recipe was compressed by means of a tableting press into biplanar tablets having a diameter of 13 mm and a mass of 600 mg.

### Example 3

Preparation of a gastroretentive system with osmotically
controlled active pharmaceutical ingredient release

| | |
|---|---|
| Ingredient | Proportion |

Active pharmaceutical ingredient layer:

| | |
|---|---|
| Active pharmaceutical ingredient | 22.5%-wt. |
| Hydroxypropyl methyl cellulose | 6.0%-wt. |
| Polyethylene oxide | 70.0%-wt. |
| Magnesium stearate | 1.0%-wt. |
| Highly dispersed silicon dioxide | 0.5%-wt. |

Osmotically active layer:

| | |
|---|---|
| Hydroxypropyl methyl cellulose | 4.0%-wt. |
| Sodium chloride | 30.0%-wt. |
| Polyethylene oxide | 65.0%-wt. |
| Magnesium stearate | 1.0%-wt. |

Coating:

| | |
|---|---|
| Cellulose acetate | 4.00%-wt. |
| Triethyl citrate | 0.14%-wt. |
| Polyethylene glycol | 2.00%-wt. |
| Acetone/isopropanol | 93.86%-wt. |
| (70:30, values in %-wt.) | |

Swelling body:

| | |
|---|---|
| Sodium alginate | 53.5%-wt. |
| Mikrocrystalline cellulose | 35.5%-wt. |
| Calcium carbonate | 1.5%-wt. |
| Crosslinked sodium carboxymethyl | 8.0%-wt. |
| cellulose | |
| Magnesium stearate | 1.0%-wt. |
| Highly dispersed silicon dioxide | 0.5%-wt. |

The components of the inner phases of active pharmaceutical ingredient layer, osmotically active layer and swelling body were granulated singly. After equipping the inner phase with the outer phase, which consisted of magnesium stearate, or of magnesium stearate and highly dispersed silicon dioxide, the active pharmaceutical ingredient layer and the osmotically active layer were compressed into a biconvex bilayered tablet, which was then provided with the coating. The coating was provided with an opening in the region of the active ingredient layer through which the active pharmaceutical ingredient can be released from this release device.

After equipping the granulate of the swelling body with the outer phase, the granulate was compressed into a tablet which was concave on one side thereof, and that side of the tablet was subsequently glued to the osmotically active layer of the release device.

### Example 4

Preparation of a gastroretentive system having erosion-controlled
active pharmaceutical ingredient release

| | |
|---|---|
| Ingredient | Proportion |

Swelling body (coating layer):

| | |
|---|---|
| Sodium alginate | 58.2%-wt. |
| Microcrystalline cellulose | 38.8%-wt. |
| Calcium carbonate | 1.5%-wt. |
| Magnesium stearate | 1.0%-wt. |
| Higly dispersed silicon dioxide | 0.5%-wt. |

Release device (core):

| | |
|---|---|
| Active pharmaceutical ingredient | 58.0%-wt. |
| Hydroxypropyl methyl cellulose | 7.5%-wt. |
| Lactose | 33.0%-wt. |
| Magnesium stearate | 1.0%-wt. |
| Highly dispersed silicon dioxide | 0.5%-wt. |

The components of the inner phases of swelling body and release device were granulated singly. After equipping the inner phase of the release device with the outer phase of magnesium stearate and highly dispersed silicon dioxide, a triangular, pointed tablet core was pressed. The granulated inner phase of the swelling body was likewise equipped with the outer phase of magnesium stearate and highly dispersed silicon dioxide. Then, part of the granulate for the coating layer of the press-coated tablet to be prepared was filled into the matrix of a tableting press, the previously prepared tablet core was placed thereon and this was topped up with the remaining granulate for the press-coated tablet, so that after compression thereof a biconvex press-coated tablet was obtained.

### Example 5

### Determining the Swelling Behaviour of Swelling Bodies

To determine the swelling behaviour of the various swelling bodies, the relative change of mass of swelling bodies that had been prepared according to Example 1 (without calcium) or Example 2 (with calcium) was determined. To this end, the swelling bodies were examined in a dissolution tester having a blade agitator, at 50 revolutions per minute in a 37° C. (±0.5° C.)-warm medium. The relative change of mass is calculated according to the formula (mₜ−m₀)/m₀, where m₀ is the mass of the swelling body at the start of the test (instant t=0) and mₜ is the mass of the swelling body following incubation in the medium for t minutes.

Initially, the swelling properties of swelling bodies were examined at constant pH value of the medium.

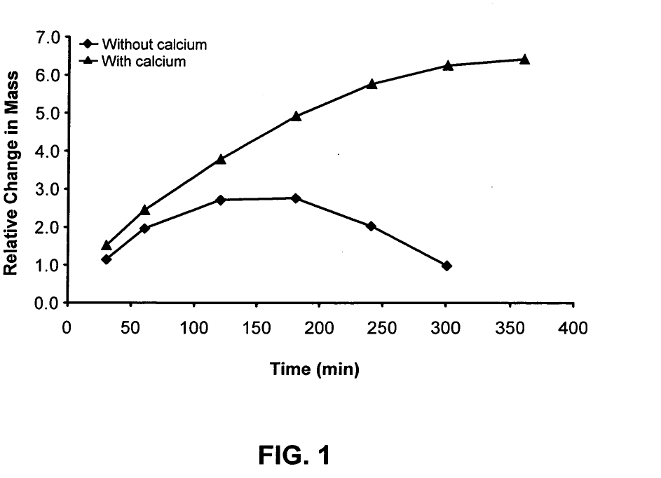

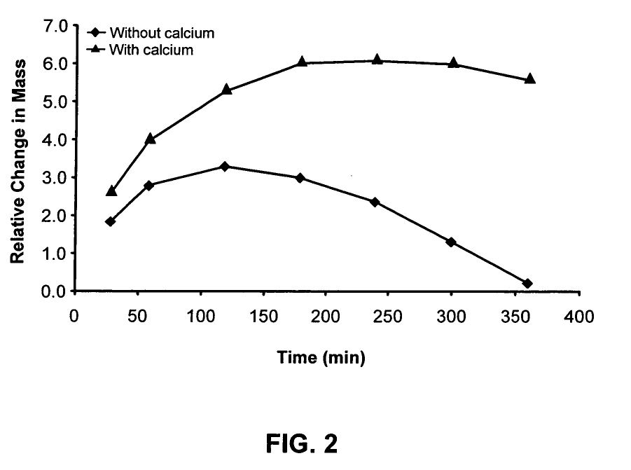

The results of these examinations are represented in the FIGS. 1 and 2. The medium either had a pH value of 3 (FIG. 1) or a pH value of 4.5 (FIG. 2). The representations of these experimental results illustrate the superior swelling properties of sodium alginate-based swelling bodies containing calcium ions, as compared to calcium-free swelling bodies. In addition, the swelling bodies comprising calcium ions exhibit an improved stability.

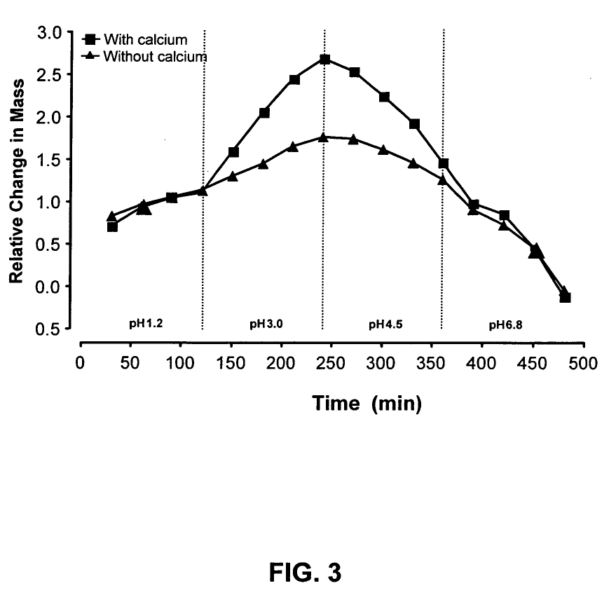

In a further series of experiments, the swelling properties of the swelling bodies were examined at varying pH values of the medium. To this end, the pH value of the medium was increased stepwise from 1.2, via 3.0 and 4.5, to 6.8. The results of these experiments are represented in FIG. 3. These results likewise show that addition of calcium ions to the sodium alginate improves the swelling properties of the swelling body. In addition, it is evident therefrom that the decomposition of swollen swelling bodies at higher pH values is not affected by the addition of calcium ions.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A gastroretentive system for the controlled, continuous release of active pharmaceutical ingredients in the stomach, comprising:
at least one release device for at least one active pharmaceutical ingredient; and
at least one swelling body that is connected to said at least one release device,
said at least one release device being an osmotic system comprising a chamber and an active pharmaceutical ingredient-containing core, an osmotically active substance in addition to the at least one active pharmaceutical ingredient, a semipermeable membrane surrounding said chamber and said core, said semipermeable membrane forming the wall of said chamber and comprising at least one outlet opening for the active pharmaceutical ingredient, and wherein said osmotic system further comprises design features for preventing direct or close contact of the outlet opening with the mucous membrane of the stomach;
or
said at least one release device being an osmotic system comprising at least two chambers within a common covering and wherein the at least one active pharmaceutical ingredient and the osmotically active substance are present in separate chambers within said common covering, and wherein the boundary of the active pharmaceutical ingredient-containing chamber is flexible at least in an area, the chamber that contains the osmotically active substance not having an opening, the chamber that contains the at least one active pharmaceutical ingredient preparation having an opening for the escape of the at least one active pharmaceutical ingredient, which opening leads outwards and penetrates said common covering, wherein after the administration of the gastroretentive system with a liquid which enters the chamber with the osmotically active substance leads to an increase in the volume of said chamber for the osmotically active substance for pushing the active pharmaceutical ingredient preparation out of said chamber for said the active pharmaceutical ingredient through the opening;
or
wherein said gastroretentive system comprises as a press-coated tablet comprising at least one swelling body and a tablet core, wherein the at least one swelling body forms a coating layer surrounding the tablet core of the press-coated tablet, said tablet core comprising an erodible mass containing at least one active pharmaceutical ingredient, and the coating layer, surrounding the tablet core having an opening for allowing the at least one active pharmaceutical ingredient to escape.

2. The gastroretentive system according to claim 1, wherein the at least one swelling body is based on a sodium alginate.

3. The gastroretentive system according to claim 2, wherein at least one of calcium ions and calcium alginate are/is added to said sodium alginate.

4. The gastroretentive system according to claim 3, wherein said calcium ions are added in the form of a pharmaceutically acceptable calcium salt selected from the group of calcium salts consisting of calcium acetate, calcium aspartate, calcium carbonate, calcium chloride, calcium citrate, calcium cyclamate, calcium folinate, calcium gluconate, calcium glutamate, calcium lactate, calcium lactate gluconate, calcium phosphate and calcium sulfate.

5. The gastroretentive system according to claim 4, wherein the proportion of calcium ions is 0.1 to 10%-wt. relative to the mass of the swelling body.

6. The gastroretentive system according to claim 2, wherein at least one of zinc ions and aluminium ions are added to the sodium alginate in a form selected from the group consisting of at least one pharmaceutically acceptable zinc salt and at least one pharmaceutically acceptable aluminium salt.

7. The gastroretentive system according to claim 6, wherein said zinc ions are added in the form of a pharmaceutically acceptable zinc salt selected from the group of zinc salts consisting of zinc acetate, zinc aspartate, zinc bishydrogen aspartate, zinc chloride and zinc gluconate, and wherein said aluminium ions are added in the form of a pharmaceutically acceptable aluminium salt selected from the group of aluminium salts consisting of aluminium hydroxide, algedrate (aluminium oxide) and aluminium phosphate.

8. The gastroretentive system according to claim 6, wherein the proportion of zinc salt or aluminium salt is between 0.1 and 30%-wt. relative to the mass of the swelling body.

9. The gastroretentive system according to claim 2, wherein the swelling body comprises a mixture of sodium alginate with at least one further polymer selected from the group of polymers consisting of croscarmellose sodium, polycarbophil, polyethylene oxide and cellulose derivatives.

10. The gastroretentive system according to claim 9, wherein the proportion of said further polymer is 1 to 30%-wt. relative to the mass of the swelling body.

11. The gastroretentive system according to claim 1, wherein said at least one swelling body comprises at least one pharmaceutically acceptable excipient selected from the group consisting of fillers, binders, flow regulators, lubricants, glidants, antiadherents and substances having an effect on the pH value.

12. The gastroretentive system according to claim 1, wherein said osmotic system comprising at least two chambers further comprises design features for preventing direct or close contact of the outlet opening with the mucous membrane of the stomach.

13. The gastroretentive system according to claim 1, wherein said design features are selected from the group consisting of hollows, concave curvatures, curved axes, angled axes, and annular shaping.

14. The gastroretentive system according to claim 1, wherein the at least one release device comprises an active pharmaceutical ingredient-containing erodible mass.

15. The gastroretentive system according to claim 1, wherein said at least one swelling body and said at least one release device are compressed with each other or glued to each other, or wherein said at least one swelling body and said at least one release device are present in a common covering.

16. The gastroretentive system according to claim 1, wherein said system is present as a press-coated tablet comprising a coating that dissolves in the stomach, or as the contents of a capsule that releases the gastroretentive system in the stomach.

17. A method for producing a gastroretentive system according to claim 1, comprising the step selected from the group consisting of gluing the at least one swelling body and the at least one release device to each other, compressing the at least one swelling body and the at least one release device with each other, and accommodating the at least one swelling body and the at least one release device in a common covering.

18. Use of a gastroretentive system according to claim 1 for the controlled administration of an active pharmaceutical ingredient.

19. The gastroretentive system according to claim 5, wherein the proportion of calcium ions is 0.3 to 8%-wt. relative to the mass of the at least one swelling body.

20. The gastroretentive system according to claim 19, wherein the proportion of calcium ions is 0.5 to 5%-wt. relative to the mass of the at least one swelling body.

21. The gastroretentive system according to claim 20, wherein the proportion of calcium ions is 0.6 to 2%-wt. relative to the mass of the at least one swelling body.

22. The gastroretentive system according to claim 8, wherein the proportion of zinc salt or aluminium salt is between 1 and 25%-wt. relative to the mass of the at least one swelling body.

23. The gastroretentive system according to claim 22, wherein the proportion of zinc salt or aluminium salt is between 5 and 15%-wt. relative to the mass of the at least one swelling body.

24. The gastroretentive system according to claim 9, wherein said cellulose derivatives are non-water-soluble cellulose derivatives.

25. The gastroretentive system according to claim 24, wherein said non-water-soluble cellulose derivatives are selected from the group consisting of ethyl cellulose and hydroxypropyl methyl cellulose.

26. The gastroretentive system according to claim 10, wherein the proportion of said further polymer is 3 to 20%-wt. relative to the mass of the swelling body.

27. The gastroretentive system according to claim 26, wherein the proportion of said further polymer is 5 to 15%-wt. relative to the mass of the at least one swelling body.

28. A gastroretentive system for the controlled, continuous release of active pharmaceutical ingredients in the stomach, comprising:
at least one release device for at least one active pharmaceutical ingredient; and
at least one swelling body that is connected to said at least one release device, said at least one release device being an osmotic system comprising a chamber and an active pharmaceutical ingredient-containing core, a semipermeable membrane surrounding said chamber and said core, said semipermeable membrane forming the wall of said chamber and comprising at least one outlet opening for the active pharmaceutical ingredient, and wherein said osmotic system further comprises design features for preventing direct or close contact of the outlet opening with the mucous membrane of the stomach;
or said at least one release device being an osmotic system comprising at least two chambers within a common covering and wherein the at least one active pharmaceutical ingredient is present in separate chambers within said common covering, and wherein the boundary of the active pharmaceutical ingredient-containing chamber is flexible at least in an area, the chamber that contains the osmotically active substance not having an opening, the chamber that contains the at least one active pharmaceutical ingredient preparation having an opening for the escape of the at least one active pharmaceutical ingredient, which opening leads outwards and penetrates said common covering, wherein after the administration of the gastroretentive system with a liquid which enters the chamber with the osmotically active substance leads to an increase in the volume of said chamber for the osmotically active substance for pushing the active pharmaceutical ingredient preparation out of said chamber for said the active pharmaceutical ingredient through the opening;
or
wherein said system is present as a press-coated tablet comprising at least one swelling body and a tablet core, wherein the at least one swelling body forms a coating layer surrounding the tablet core of the press-coated tablet, said tablet core comprising an erodible mass containing at least one active pharmaceutical ingredient, and the coating layer, surrounding the tablet core having an opening for allowing the at least one active pharmaceutical ingredient to escape.
